(19) United States
(12) Patent Application Publication
(10) Pub. No.: US 2002/0177808 A1
(43) Pub. Date: Nov. 28, 2002

(54) Mechanism for prevention of premature activation

(75) Inventor: Udi Carmel, Kiryat Ono, IL (US)

Correspondence Address: CAESAR, RIVISE, BERNSTEIN, COHEN & POKOTILOW, LTD., ATTN: ELAN, 12TH FLOOR, SEVEN PENN CENTER, 1635 MARKET STREET, PHILADELPHIA, PA, 19103-2212, US

(73) Assignee: ELAN PHARMA INTERNATIONAL LIMITED, Shannon Business Park (IE)

(21) Appl. No.: 10/150,229

(22) Filed: May 17, 2002

Related U.S. Application Data
(60) Provisional application No. 60/292,647, filed on May 22, 2001.

(51) Int. Cl. A61M 1/00

(52) U.S. Cl. 604/124

(57) ABSTRACT

A mechanism and method for preventing premature activation of an action includes a safety device. The safety device includes a housing that is arranged to enable such an action when the housing is in a desired orientation (e.g., horizontal, vertical). The housing includes a bowl having a concave cavity, a ball located in the cavity and a trigger having an actuator and a sleeve arranged to slide about the ball when the ball is in a predetermined position of the cavity to move the actuator and initiate the action.

### RELATED APPLICATIONS

This application is a utility application based on Provisional Application Serial No. 60/292,647, filed May 22, 2001, entitled MECHANISM FOR PREVENTION OF PREMATURE ACTIVATION.

### FIELD OF THE INVENTION

This invention relates to the prevention of premature activation of an action, and in particular, to a mechanism that prevents premature activation of the action (e.g., administration and/or delivery of a drug into a living organism, such as a human body or an animal body).

### BACKGROUND OF THE INVENTION

Prevention of a premature activation of an action is critical when the action would not be successful if started before a condition or set of conditions is satisfied. One area where the prevention of a premature activation of an action is especially useful is in the field of delivery of medications. For example, if an action of drug delivery is activated prematurely, then depending on the delivery device, the drug may not eject as desired from the device or the drug may not be delivered to a desired delivery site. Moreover, if the device is not oriented properly and the device is activated prematurely, the drug may be injected with air bubbles therein which may be extremely harmful or fatal to the patient.

Previously, various devices have been developed for the delivery of medications into and through the skin of living organisms. These devices include syringes in which a liquid drug solution is delivered through the skin of a user from a syringe chamber by the manual movement of a syringe plunger to move the drug solution from the chamber through the syringe needle inserted under the skin. While some of these devices may have included safety mechanisms (e.g., caps, covers) for preventing the premature activation of an action (e.g., drug delivery), no safety mechanisms were known that prevent or allow the activation based on the three-dimensional angular orientation (e.g., horizontal, vertical) of the various devices.

The liquid drug solution can be a mixture of a drug (e.g., powdered, lyophilized, concentrated liquid) and a diluent (e.g., dextrox solution, saline solution, water), since certain injectable substances (e.g., glycogen) do not maintain their chemical and physical stability when mixed with a diluent and thus cannot be stored for a substantial period of time. Therefore, powdered, concentrated or lyophilized substances (e.g., drugs or compounds) are presently used for injection of materials that would otherwise be unstable. Lyophilization, for example, is the rapid freezing of a material at a very low temperature followed by rapid dehydration by sublimation in a high vacuum. The resulting lyophilized substance is typically stored in a glass vial or cartridge which is closed by a cap, such as a rubber stopper or septum. Some liquid drug solutions may also be stored as a liquid arranged for delivery without lyophilization and reconstitution.

It is generally necessary to reconstitute the concentrated or solid material (e.g., lyophilized substance) if it is to be delivered as a liquid. Reconstitution, for example, is accomplished by mixing the concentrated or solid substance with a suitable diluent or liquid. Reconstitution typically involves the use of a syringe with a needle to inject the diluent from a first vial into the vial containing the substance. The substance is then thoroughly mixed to form the drug solution, for example, by swirling the vial by hand. Typically, the drug solution is then forwarded through the needle into the injection site.

It is preferable that delivery systems having reservoirs and drug vials are positioned so that the drug vials are positioned vertically. Vertical orientation of the vials is preferred so that the system can perform optimally (e.g., to prevent drug blocking, to insure delivery within a predetermined duration, and to prevent air from getting into the delivery channels or passageways). Furthermore, it is difficult to insure that the injection system is positioned as desired (e.g., vertically or horizontally) when the person injecting the drug does not have eye contact with the injector. Therefore, it would be beneficial if a delivery system would inject the drug only when positioned in its preferred orientation during injection.

It is desirable to have a simple, reliable system that facilitates safe preparation and delivery of a reconstituted compound. In addition, it is desirable to provide a system that reconstitutes a lyophilized drug while maintaining sterility throughout the process. Further, it is desirable to provide a delivery system (e.g., syringe) that facilitates safe delivery of a liquid drug. Also, it is desirable to provide improvements in the subcutaneous delivery of medication generally, by preventing drug blocking, insuring delivery within a predetermined duration, and preventing air from getting into delivery passageways, which provides for a safe, effective administration by the user. Moreover, it is desirable to provide a system that reduces needle phobia.

### SUMMARY OF THE INVENTION

In a preferred embodiment, a safety device includes a housing, a trigger within the housing, and an orientation unit in communication with the housing and the trigger. The trigger is movable from a first position to an activation position. When the trigger is in the activation position, the trigger initiates the action. The orientation unit enables movement of the trigger from the first position to the activation position only when the housing is in a predetermined orientation in all three dimensions.

In another preferred embodiment, the system is a fluid delivery device for delivering a drug to the user only when the system is in a preferred orientation. The delivery device comprises a housing, a trigger within the housing, an orientation unit in communication with the housing and the trigger, and an injection assembly within said housing and in communication with said trigger. The trigger is movable from a first position to an activation position. When the trigger is in the activated position, the trigger initiates the action. The orientation unit enables movement of the trigger from the first position to the activated position only when the housing is in a predetermined three dimensional orientation in all three dimensions. The injection assembly is held under a releasable bias and arranged to expel the fluid from the delivery device upon release of the bias.

In some examples of the preferred embodiments, the orientation unit may include an indicator arranged for providing an indication that the housing is in the predetermined orientation in all three dimensions. The orientation unit may also include a cavity defined by an inner rim, the indicator freely moveable within the cavity in response to the orientation of the housing relative to the vertical or horizonal, the indicator moving to a desired position of the cavity as the indication that the housing is in the predetermined orientation in all three dimensions. Further, the trigger may be arranged to move to the activating position only when the indicator is in the desired position. The indicator could be a ball having a diameter.

In these examples of the preferred embodiments, the trigger is preferably slidingly located within the housing, the trigger preferably including a hollow sleeve sitting adjacent the inner rim of the cavity, the hollow sleeve having an inner diameter greater than a transverse dimension of the indicator, the sleeve arranged to slide about the indicator when the indicator is in the desired position to move the trigger to initiate the action. The indicator is preferably a ball.

In yet another preferred embodiment, a method of ejecting a fluid from a delivery device is disclosed. The delivery device includes a housing, a trigger, an orientation unit, and an injection assembly. The housing is arranged for injecting a fluid into an injection site. The trigger is located within the housing, and the orientation unit is in communication with the housing and the trigger. The injection assembly is located within the housing and in communication with the trigger. The injection assembly is held under a releasable bias and arranged to eject the fluid from the delivery device upon release of the bias. The method includes enabling movement of the trigger from a first position to an activation position only when the housing is in a predetermined orientation in all three dimensions, activating the injection assembly by releasing the bias by the movement of the trigger, and providing an egress for ejecting the fluid from the delivery device.

Further scope of applicability of the present invention will become apparent in the description given hereinafter. However, it should be understood that the detailed description and specific examples, while indicating preferred embodiments of the invention, are given by way of illustration only, since the invention will become apparent to those skilled in the art from this detailed description.

### BRIEF DESCRIPTION OF THE DRAWINGS

The invention will be described in conjunction with the following drawings, in which like-referenced numerals designate like elements, and wherein:

FIG. 1 is an exploded isometric view of a drug delivery device in accordance with a first preferred embodiment of the invention;

FIG. 2 is a top view of the drug delivery device of FIG. 1;

FIG. 3 is a sectional view of the drug delivery device taken along line 3-3 of FIG. 2;

FIG. 4 is a side view of the drug delivery device shown in FIG. 1 shown without a safety tab for clarity;

FIG. 5 is a top view of the drug delivery device of FIG. 4;

FIG. 6 is a sectional view of the drug delivery device taken along line 6-6 of FIG. 4;

FIG. 7 is a sectional view of the drug delivery device taken along line 7-7 of FIG. 4;

FIG. 8 is a top view of the bottom cover of the drug delivery device shown in FIG. 1;

FIG. 9 is a sectional view of the drug delivery device taken along line 9-9 of FIG. 5 shown in a tilted position;

FIG. 10 is a sectional view of the drug delivery device taken along line 10-10 of FIG. 4;

FIG. 11 is a view similar to that of FIG. 10, but showing the drug delivery device shown in a tilted position;

FIG. 12 is a sectional view of the drug device taken along line 12-12 of FIG. 4;

FIG. 13 is a view similar to that of FIG. 12, but showing the drug delivery device in an injection state;

FIG. 14 is a sectional view similar to that of FIG. 3, but showing the drug delivery device in an injection state; and

FIG. 15 is a sectional view similar to that of FIG. 14, but showing the drug delivery device in a post-injection state.

### DETAILED DESCRIPTION OF THE INVENTION

The present invention is directed to a premature activation prevention mechanism and a method for preventing premature activation of an action (e.g., delivering a drug in solution under pressure, transmitting a signal, actuating a switch). The premature activation prevention mechanism is a safety device that prevents a premature action, and will be described in greater detail below. The mechanism includes a housing that is arranged to enable such an action. The housing includes a bowl having a concave cavity, a ball located in the cavity and a trigger having an actuator and a sleeve arranged to slide about the ball when the ball is in a predetermined position of the cavity to move the actuator to initiate the action.

For purposes of illustration, the premature activation prevention mechanism will be shown in combination with a drug delivery device. Of course, the premature activation device can be used on any device, not limited to drug delivery devices, where it is desired to have a safety mechanism that prevents activation unless the device is situated at a desired angle.

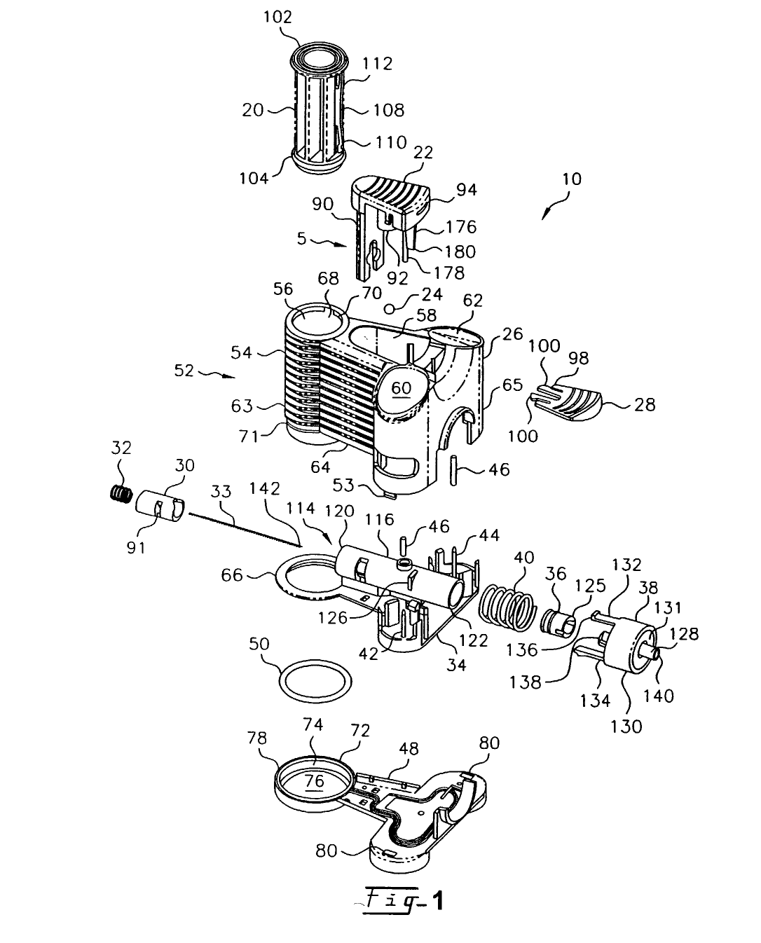

Referring to FIG. 1, a drug delivery device having a premature activation prevention mechanism 5 is schematically illustrated at 10. The delivery device 10 includes a piston 20, a trigger 22, a ball 24, a body 26, a safety tab 28, a hub 30, a hub spring 32, a needle 33, a chassis 34, a septum 36, a shield 38, a shield spring 40, a liquid vial spike 42, a drug vial spike 44, spike sleeves 46, a bottom cover 48, and an O-ring 50. In this exemplary delivery device 10, the body 26, chassis 34 and bottom cover 48 form a housing 52. However, the housing 52 is generally arranged to include structural elements for performing an action (e.g., delivering a drug, sending a signal). The structural elements described in this preferred embodiment can be formed of any suitable material, e.g., plastic, metal, rubber.

Referring to FIGS. 1 through 15, and in particular to FIG. 1, the body 26 includes several cavities and downward extending tabs 53. In particular, the body 26 includes a piston barrel 54 having a piston barrel cavity 56 arranged for receiving the piston 20, a trigger receiving cavity 58 arranged for receiving the ball 24 and trigger 22, a liquid vial receiving cavity 60 arranged for receiving a liquid vial (conventional or otherwise), and a drug vial receiving cavity 62 arranged for receiving a drug vial (conventional or otherwise). The body 26 also includes a bottom portion 64 that is hollow to receive the chassis 34 and to connect with the bottom cover 48. As shown, the piston barrel 54 is located at a proximal end 63 of the device 10, and the vial receiving cavities 60, 62 are located at a distal end 65 of the device 10.

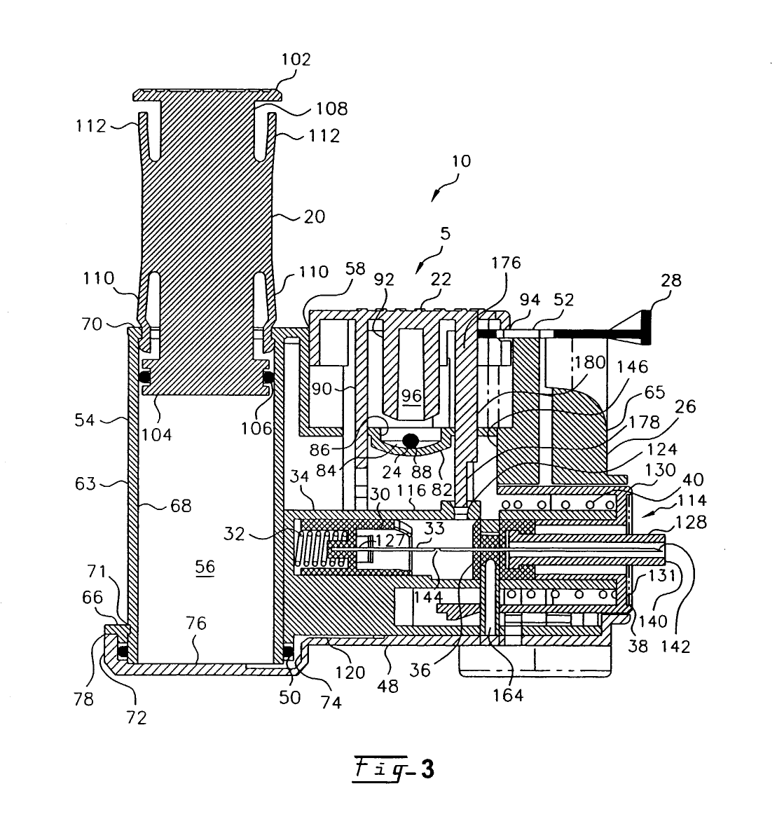

Referring in particular to FIGS. 1 and 3, the chassis 34 includes a chassis ring 66. The body 26 is attached to the chassis 34 by sliding the piston barrel 54 of the body 26 through the chassis ring 66. The piston barrel 54 has an inner wall 68 defining the cavity 56. The inner wall 68 extends from a lower end of the barrel to an upper lip 70 of the barrel 54. The piston barrel 54 also has a slot 71 at its lower end for snap fitting the chassis ring 66. The vial spikes 42, 44 extend into the vial receiving cavities 60, 62 of the body 26, respectively.

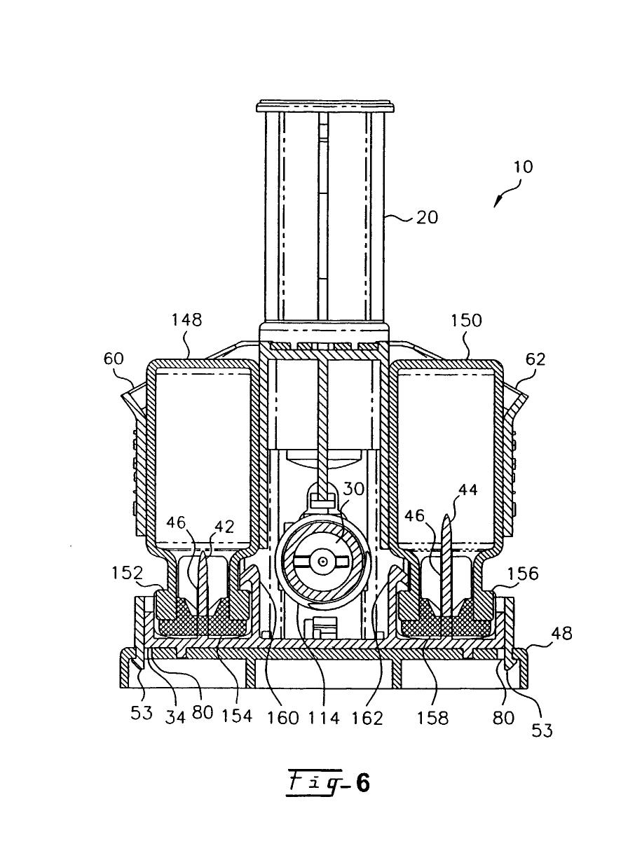

The bottom cover 48 includes a well 72 having a circular wall 74 extending up from a floor 76 and terminating at an upper rim 78. The bottom cover 48 also includes slots 80 arranged to accept the downward extending tabs 53 of the body 26. The body 26 and chassis 34 are connected to the bottom cover 48 by attaching the piston barrel 54 and chassis ring 66 to the circular wall 74 and upper rim 78, and snap fitting the tabs 53 of the body 26 into the slots 80 of the bottom cover 48. In this embodiment, the O-ring 50 is located between the chassis ring 66, piston barrel 54, and circular wall 74 of the well 72 to prevent fluid leakage through the interface. The integration of the body 26, chassis 34 and bottom cover 48 into the housing 52 is best shown in FIGS. 3 and 6, which will be described in greater detail below.

The premature activation prevention mechanism 5 is a subassembly of the delivery device 10. As seen in FIGS. 1, 3 and 9-15, the premature activation prevention mechanism 5 includes the trigger 22, ball 24, and a bowl 82 arranged in the housing 52. The housing 52, and in particular the body 26, comprises the bowl 82, which includes a concave cavity 84 defined by an inner rim 86. The bowl 82 is arranged to accept the ball 24 in its cavity 84 such that the ball 24 can rotate or slide within the cavity 84 based on the angular orientation of the housing 52, and in particular, of the bowl 82. In this preferred embodiment, the ball 24 sits at the bottom 88 of the cavity 84 when the housing 52 is horizontal. However, the angular relationship between the bowl 82 and the housing 52 can be designed so that the ball 24 sits at the bottom 88 of the cavity 84 when the housing 52 is in desired orientation (e.g., vertical, horizontal) as readily understood by a skilled artisan.

The preferred shape of the cavity 84 is concave. The concave shape of the cavity 84 can vary substantially, for example, from relatively flat to hemispherical depending on how much of a range of angular orientation the bowl 82 or housing 52 can vary from the predetermined angular orientation (e.g., horizontal, vertical) and permit the hollow sleeve 92 to slide about the ball. For example, the concavity of the cavity 84 can be based on a mathematical formula, where the concavity is lower (e.g., closer to flat, substantially flat) when it is more important for the bowl 82 or housing 52 to be about (e.g., within five or ten degrees of) the predetermined angular orientation for injection. Likewise the concavity can be greater (e.g., closer to semispherical or frusto-conical) when a greater range (e.g., 60 degrees) of orientation is allowable for injection.

The ball 24 is preferably formed of a hard or semi-hard material (e.g. metal, plastic) that can easily roll or slide around within the concave cavity 84. The ball 24 preferably has a spherical shape. However, the ball 24 can have any shape or be formed of any material that allows it to roll or slide about within the cavity 84, as readily understood by a skilled artisan.

As best shown in FIG. 3, the trigger 22 is slidingly located within the trigger receiving cavity 58 of the body 26. The trigger 22 includes an actuator 90, a hollow sleeve 92 and a slot 94. As shown in FIGS. 3, 9-11,14 and 15, the sleeve 92 sits atop the inner rim 86 of the bowl 82 and extends towards the cavity 84. At its distal end, the hollow sleeve 92 comprises a sleeve cavity 96 having an inner diameter greater than the diameter of the ball 24 and can slide down and about the ball 24 when the ball 24 is in a desired position (e.g., center, lowest point) of the cavity 84. The actuator 90 slides through a slot 91 of the hub 30 and a slot 93 of a needle barrel 116, as will be described in more detail below.

As can best be seen in FIGS. 1 and 3, the safety tab 28 is slidingly locatable into the slot 94 of the trigger 22. In this example of the preferred embodiment, the safety tab 28 includes two distal fingers 98 that are biased inwardly to slide through the trigger 22 slot. The distal fingers 98 have enlarged ends 100 that snap fit in the slot 94 to prevent a premature movement of the trigger 22. In other words, placing the distal fingers 98 of the safety tab 28 through the trigger slot 94 prevents the trigger 22 from sliding longitudinally into the housing 52 and actuating a drug delivery. The safety tab 28 can be removed from the trigger 22 by pulling the tab 28 away from the trigger 22 so that the distal fingers 98 are disengaged from the trigger slot 94.

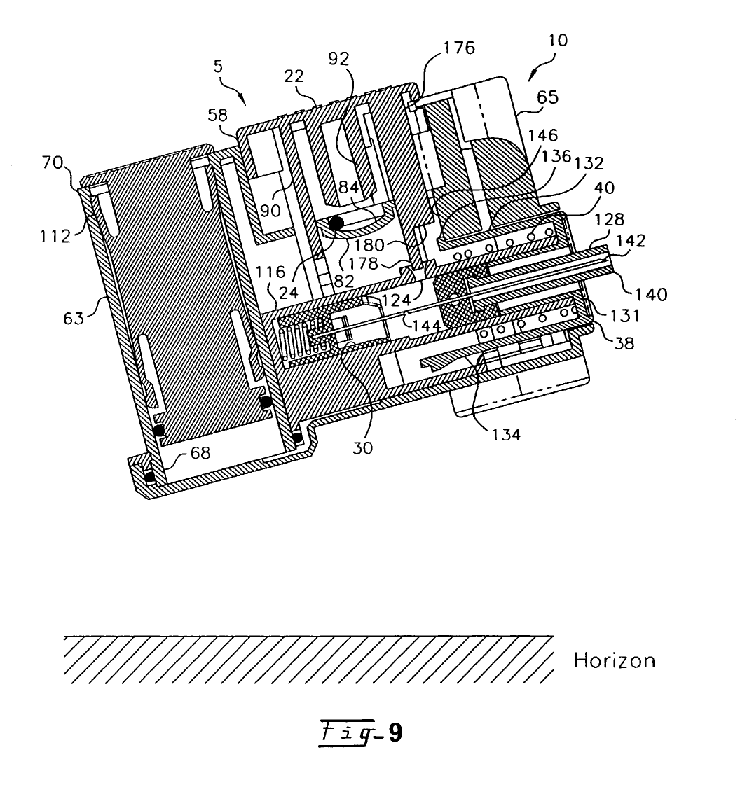

As can best be seen in FIGS. 3, 9,14 and 15, the piston 20 is slidingly located within the piston barrel 54. The piston 20 includes a handle 102 at its proximal end and a plunger 104 at its distal end. The plunger 104 includes an O-ring or outer rim 106 having an outer diameter just slightly greater than the inner diameter of the piston barrel 54 to form a sliding seal therewith so that no fluid can gain egress through the interface of the plunger 104 and the piston barrel 54. Alternatively, the plunger 104 can be formed of an elastomeric material and have an outer diameter slightly greater than the inner diameter of the piston barrel 54. Either construction is preferred because the plunger 104 makes sliding frictional engagement with the inner wall 68 of the piston barrel 54 for pushing or pulling a fluid out of or into the barrel 54. The plunger 104 thus makes sliding frictional engagement with the inner wall 68 of the piston barrel 54 for pushing or pulling a fluid out of or into the cavity 56 of the piston barrel 54.

The piston 20 also includes a rod 108 extending from the plunger 104 to the handle 102 of the piston 20. The rod 108 includes two sets of fingers. The first set of fingers 110 extend from a central region of the rod 108 toward the plunger 104. As best seen in FIG. 3, when the piston 20 is in a retracted position within the piston barrel 54, the first set of fingers 110 are radially biased outward towards the inner wall 68 of the barrel 54 and the fingers 110 are snap fitted against the upper lip 70 of the inner wall 68.

The second set of fingers 112 extend from the central region of the rod 108 toward the handle 102 of the piston 20. As can best be seen in FIGS. 9, 14 and 15, when the piston 20 is in a depressed position, the fingers 112 are radially biased outward toward the inner wall 68 of the piston barrel 54. In this position, the fingers 112 are snap fitted under the upper lip 70 of the inner wall 68 to lock the piston 20 in this position.

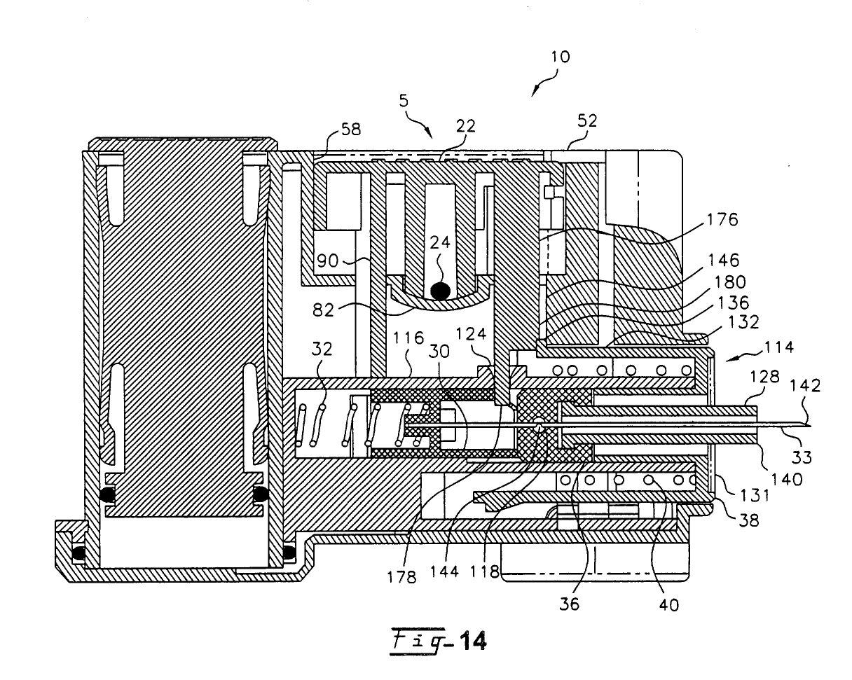

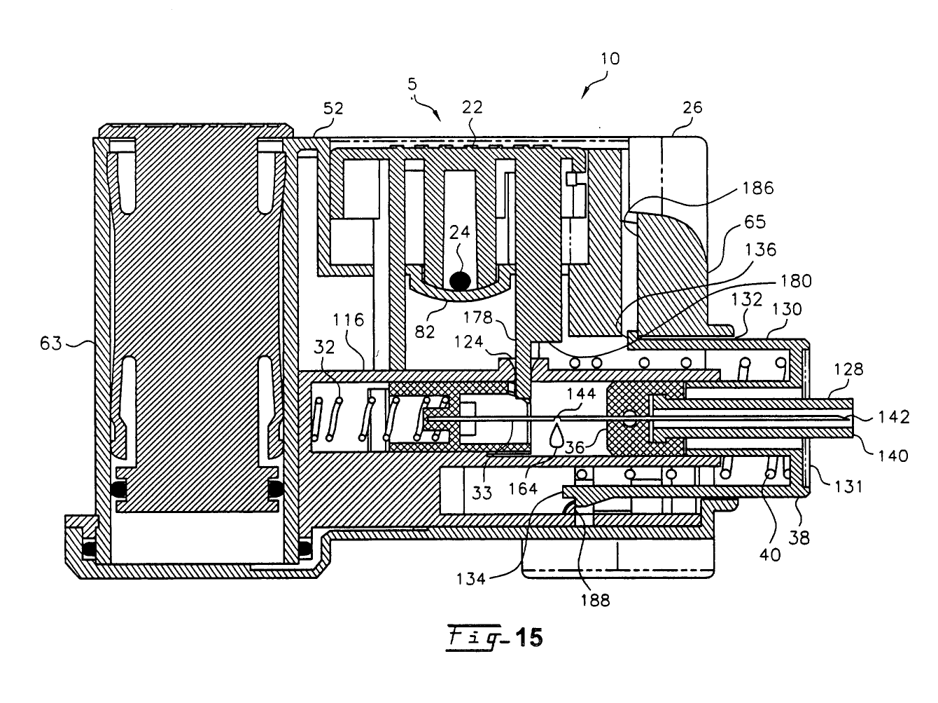

Still referring in general to FIG. 1, the drug delivery device 10 includes an injection assembly 114 within the housing 52. Prior to injection, the injection assembly 114 is held under a bias and is arranged to expel a drug solution from the delivery device 10 upon release of the bias. The injection assembly 114 is best seen in FIGS. 3,9,14 and 15. FIGS. 3 and 9 are sectional views showing the assembly 114 in its pre-injection position. FIGS. 14 and 15 are sectional views showing the injection assembly 114 in its injection position and post-injection position, respectively.

The injection assembly 114 basically comprises a needle barrel 116, the hub 30, the hub spring 32, the septum 36, the shield 38, and the shield spring 40. The needle barrel 116 comprises a hollow cylindrical axial tube 118 that receives the hub 30, hub spring 32 and needle 33 at its proximal end 120 and that receives the septum 36 and shield 38 at its distal end 122. As can best be seen in FIG. 1, the needle barrel 116 also comprises an opening 124 through the tube 118, a flange 126 extending radially outward from the tube 118, and a slot 93 arranged for slidingly receiving the actuator 90. The opening 124 is arranged for permitting a trigger pin 176 to slide therethrough into the hollow interior of the needle hub 30. The flange 126 is arranged for abutting the shield spring 40, as will be described in greater detail below.

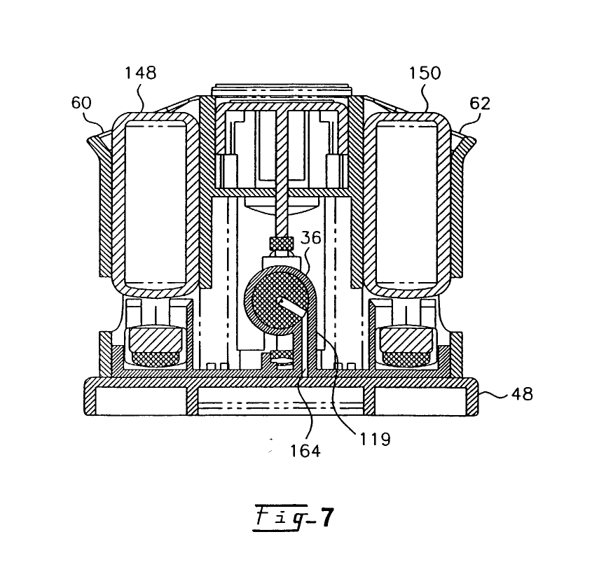

The hub 30 comprises a slot 91 to slidingly receive the actuator 90, and further comprises a cup shaped upper section having a centrally located bore 127. The bore 127 mounts about and holds the needle 33, which extends within the needle barrel 116 through the septum 36. The septum 36 is preferably formed of rubber and defines an injection chamber 119 (FIG. 7) used for communicating a drug solution from the housing 52 to the interior of the hollow needle 33. The septum 36 has a cylindrical body and a cup shaped distal end 125 arranged to be attached to the shield 38. Preferably the cup shaped distal end 125 is slotted so as to more easily receive an inner sleeve 128 of the shield 38, which will be described in more detail below. As shown in FIGS. 3, 9,14 and 15, the septum 36 is slidingly engaged within the needle barrel 116. The septum 36 preferably has an outer diameter slightly greater than the inner diameter of the needle barrel 116 and forms a sliding seal therewith so that no fluid can gain egress through the interface of the septum 36 and the needle barrel 116.

The shield 38 includes the inner sleeve 128 and a cup shaped outer sleeve 130 slidingly engaged at the distal end 65 of the housing 52. The shield 38 has an outer face 131 arranged to be placed against the injection site, as will be described in greater detail below. Extending longitudinally from the outer sleeve 130 are an upper arm 132 and a lower arm 134. The upper arm 132 has a hook 136 that extends radially outward to communicate with the body 26, as shown by example in FIGS. 9,14 and 15. The arms 132, 134 are flexible so as to move radially upon the application of an external force and will be described in greater detail below. The inner sleeve 128 extends within the outer sleeve 130 and terminates at two opposite ends: a septum coupling end 138 and a needle covering end 140. The inner sleeve 128 is adapted to be slidingly located within the needle barrel 116. The septum coupling end 138 of the inner sleeve 128 is arranged to couple to the septum 36 and to slidingly receive the needle 33.

The injection needle 33 has a proximal end connected to the needle hub 30, a distal sharp end 142, and a notch 144. In the pre-injection position the distal sharp end 142 of the needle 33 extends close to but not beyond the needle covering end 140 of the inner sleeve 128, as can best be seen in FIGS. 3 and 9. Also in this pre-injection position, the hook 136 of the upper arm 132 abuts a first retaining wall 146 of the housing 52 to keep the shield 38 in its pre-injection position and holds the shield spring 40 in its compressed state. The shield spring 40 is a helical compression spring located within the interior of the outer sleeve 130 immediately adjacent the needle barrel 116 between a distal end of the shield 38 and the flange 126 extending radially from the tube 118 of the needle barrel 116 (FIGS. 1, 3, 9 and 15).

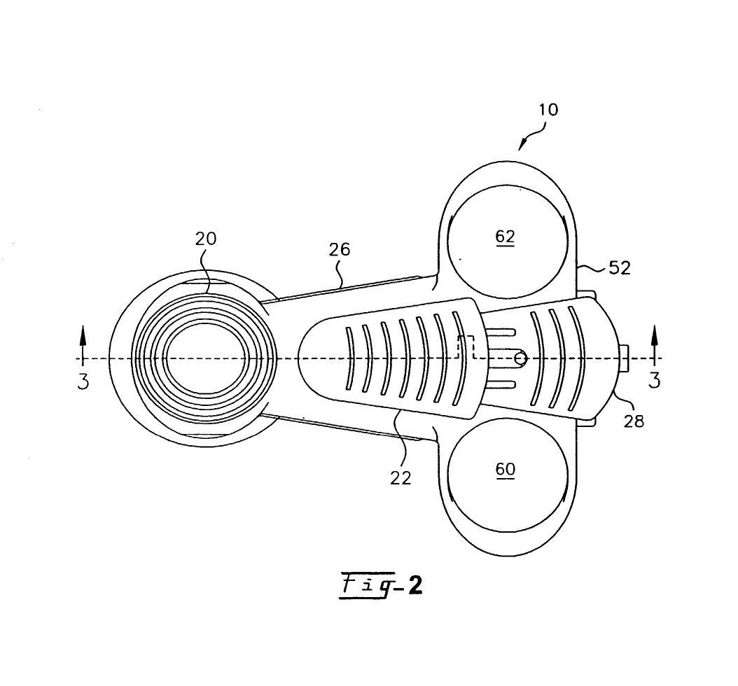

The elements of the delivery device 10 described above and shown in FIG. 1 are illustrated in FIGS. 3-15 during different stages of a drug delivery. FIG. 2 shows a top view of the delivery device 10 at an initial stage prior to delivery of a drug solution. FIG. 3 is a longitudinal section view showing the drug delivery device 10 taken along line 3-3 of FIG. 2. As shown in FIG. 3, the piston 20 is temporarily held in place by its first set of fingers 110 against the upper lip 70 of the piston barrel 54. In this position, the plunger 104 of the piston 20 is near the upper lip 70.

Still referring to FIG. 3, the safety tab 28 is shown engaged through the slot 94 of the trigger 22 to prevent the trigger 22 from prematurely sliding down into the housing 52 and activating the delivery. Moreover, in this initial stage, both the hub spring 32 and the shield spring 40 are in a compressed state and the distal sharp end 142 of the needle 33 is not exposed. The ball 24 is located at the bottom 88 of the cavity 84 of the bowl 82, indicating that the delivery device 10 is in a horizontal orientation.

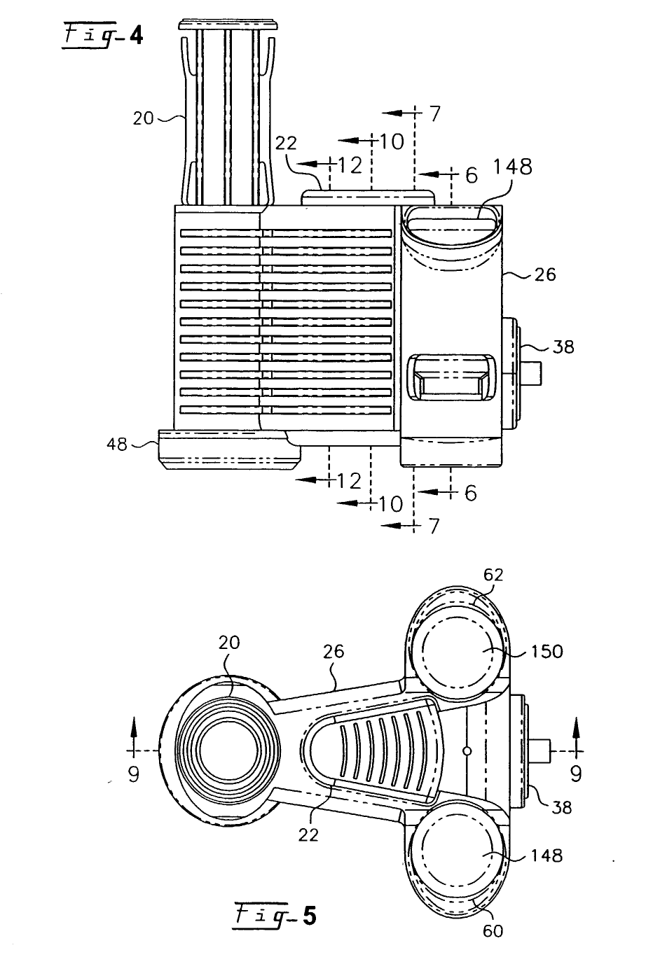

FIGS. 4 and 5 illustrate side and top views of the drug delivery device 10 at a second stage. This second stage is a pre-injection stage different than the initial stage because the safety tab 28 has been removed from the trigger 22. In addition, a liquid vial 148 and a drug vial 150 have been inserted into the liquid vial receiving cavity 60 and the drug vial receiving cavity 62, respectively, of the body 26. The vials 148,150 can readily be seen in FIG. 6, which is a sectional view of the delivery device 10 taken along line 6-6 of FIG. 4. The liquid vial 148 contains a liquid (e.g., diluent) and comprises an accessible end 152 plugged by a rubber or plastic stopper 154. Similarly, the drug vial 150 contains a drug and includes an accessible end 156 plugged by a rubber or plastic stopper 158. Preferably, the drug in the drug vial 150 is powdered or lyophilized and requires reconstitution, rehydration or dilution for delivery. Alternatively, the drug may be in a fluid form.

The vial spikes 42, 44 bring the interior of the vials 148, 150 in fluid communication with the delivery device 10. The liquid vial spike 42 penetrates the stopper 154 of the liquid vial 148 when the vial 148 is inserted into the liquid vial receiving cavity 60. As shown in FIG. 6, the liquid vial 148 is held in the cavity by a latch 160 coupled to the chassis 34. Preferably, a spike sleeve 46 is located between the spike 42 and the stopper 154 to allow the liquid vial spike 42 to be sealably and slidably moveable with the stopper 154. Both vial spikes 42,44 have two paths extending longitudinally within the spikes for communication between the respective vial 148,150 and the delivery device 10. Examples of vial spikes having two paths extending longitudinally within are disclosed in U.S. patent application Ser. No. 09/439,963, commonly owned, whose disclosure is incorporated by reference herein in its entirety.

The liquid vial spike 42 includes an air inlet and a liquid outlet as its two longitudinally extending paths. The air inlet is adapted to provide fluid communication between the piston barrel cavity 56 and the interior of the liquid vial 148. The liquid outlet is arranged for providing fluid communication between the interior of the liquid vial 148 and the drug vial spike 44. Once the liquid vial 148 is inserted through the liquid vial receiving cavity 60, pressurized air enters the liquid vial 148 from the piston barrel cavity 56 and forces the liquid out of the liquid vial 148 to the drug vial 150, as further described below.

The drug vial spike 44 penetrates the stopper 158 of the drug vial 150 when the vial 150 is inserted into the drug vial receiving cavity 62. Like the liquid vial 148, the drug vial 150 is held in the cavity by a latch 162 coupled to the chassis 34. Preferably, a spike sleeve 46 is located between the drug vial spike 44 and the stopper 158 to allow the drug vial spike 44 to be sealably and slidably moveable with the stopper 158.

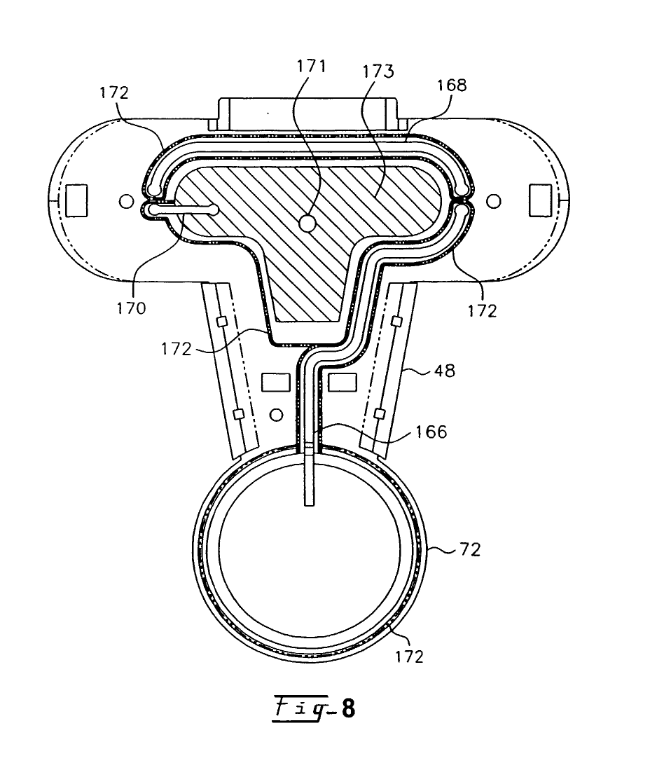

The drug vial spike 44 includes a liquid inlet and a drug outlet as its two longitudinally extending paths. The liquid inlet is adapted to provide fluid communication between the liquid vial 148 and the interior of the drug vial 150. The drug outlet is arranged for providing fluid communication between the interior of the drug vial 150 and the injection assembly 114. Once the drug vial 150 is inserted, pressurized liquid from the liquid vial 148 enters the drug vial 150. The pressurized liquid is mixed with the drug to form the drug solution, which is forced by the pressure toward the injection assembly 114, as can best be seen by FIGS. 7 and 8.

FIG. 7 is a sectional view taken along line 7-7 of FIG. 4. Of particular interest is a drug inlet 164 which provides fluid communication between the drug vial 150 and the injection chamber 119 of the septum 36, described in greater detail below. The drug inlet 164 can also be seen in FIG. 3. The injection chamber 119 communicates between the drug inlet 164 and the needle 33.

The bottom cover 48 is best seen in FIG. 8. The bottom cover 48 includes a fluid channel 166, a liquid channel 168, a drug solution channel 170, a channel opening 171, and a hydrophilic membrane 173 for communicating with the drug inlet 164. The fluid channel 166 communicates between the well 72 and the fluid inlet of the liquid vial spike 42, the liquid channel 168 communicates between the liquid outlet of the liquid vial spike 42 and the liquid inlet of the drug vial spike 44, and the drug solution channel 170 communicates between the drug outlet of the drug vial spike 44 and the channel opening 171 via the hydrophilic membrane 173.

The hydrophilic membrane 173 is a layer of known fiber (e.g., polymer such as nylon, polypropylene) that creates a capillary effect. The hydrophilic membrane 173 allows liquid to flow therethrough, but blocks the passage of air. However, in this example of the preferred embodiment, in order to block the passage of air, the membrane 173 must first be wet. That is, air will flow through a dry membrane 173, but will not flow through a wet membrane 173. Accordingly, as best seen in FIG. 8, after membrane gets wet from the liquid drug pushed out of the drug vial 150, the membrane 173 allows the liquid drug through but blocks air. In addition, once the membrane 173 is wet, then any subsequent air pushed into the membrane 173 also blocks any further liquid from passing through. Therefore if an air pocket reaches the membrane 173 before all the liquid drug has been delivered, then the membrane 173 will block passage of both the air pocket and the subsequent liquid drug. This is another reason why it is critical for the drug delivery device 10 to be substantially horizontal in this example. If the delivery device 10 is substantially horizontal, then once the membrane 173 is wet, air will not flow out of the drug vial 150 into the membrane 173 until a desired amount of the drug has been injected.

Referring to FIGS. 6-8, the piston 20 is pushed into the piston barrel cavity 56, which pushes the air in the piston barrel cavity 56 through the fluid channel 166 to the liquid vial 148. The air pushed from the piston barrel cavity 56 into the liquid vial 148 increases pressure within the vial 148. The increased pressure pushes diluent out of the liquid vial 148 through the liquid channel 168 and into the drug vial 150, where it is mixed with the drug in the drug vial 150 under the increased pressure to form the drug solution. If the delivery device 10 is substantially horizontal, the drug solution is pushed by the increased pressure through the drug solution channel 170 and the hydrophilic membrane 173 into the drug inlet 164 where it is contained under pressure.

A weld line 172 (FIG. 8) is formed around the channels 166, 168, 170 to prevent leakage between the interface of the channels and the chassis 34. In other words the weld line 172 ensures that the respective fluids (e.g., air, diluent, liquid drug solution) flow along their respective channels 166,168,170, and do not leak into an undesired channel or area. The weld line 172 seals the bottom cover 48 to the chassis, preferably by ultrasonic or heat welding. The latches 160, 162 secure the liquid and drug vials 148, 150 to the delivery device 10 during this mixing process.

In this preferred embodiment, air from the piston barrel cavity 56 is forced into the fluid channel 166 to pressurize the drug delivery device 10. It is understood that a fluid or liquid could also be used to pressurize the device 10 by using a fluid or liquid instead of air. It is also understood that the type of fluid, diluent or drug contained in the vials 148, 150 or in the piston barrel cavity 56 is not critical to this preferred embodiment of the invention as long as the trigger mechanism prevents premature activation of the desired action, for example, drug delivery.

The depression of the trigger 22 displaces the injection needle 33 which is used to inject the reconstituted drug solution into a user tissue. However, as can best be seen in FIGS. 3, 9-11 and 14, a critical feature of this invention is that the trigger 22 can only be pressed to activate the injection when the delivery device 10, and in particular the bowl 82, is in a predetermined (e.g., horizontal) orientation. In other words, the ball 24 must be located in the bowl 82 in a position that permits the hollow sleeve 92 to slide down around the ball 24. If the ball 24 is not in this position, the sleeve 92 cannot activate the delivery action of the device 10 because the ball 24 blocks the sleeve 92 from sliding around the ball 24 to place the trigger 22 in an activation position. In this preferred embodiment, the ball 24 must be centered in the cavity 84 of the bowl 82 so that the sleeve 92 can slide around the ball 24 and activate the drug delivery. In this example of the preferred embodiment, the delivery device 10 must be substantially horizontal for the trigger 22 to activate delivery.

In order to more fully disclose the relationship between the ball 24 and the hollow sleeve 92, FIG. 9 shows a sectional view of the delivery device 10 taken along line 9-9 of FIG. 5. As shown, the delivery device 10 is not horizontal. In fact, the distal end 65 of the delivery device 10 is higher than the proximal end 63. In this orientation, the ball 24 is located at a back end of the bowl 82. With the ball 24 in this location, the trigger 22 does not slide down sufficiently far enough to activate the delivery because the ball 24 interferes with the downward movement of the trigger 22.

More particularly, if the exemplary delivery device 10 is not at least substantially horizontal in three dimensions, the ball 24 will rest at least far enough away from the bottom of the cavity 84 of the bowl 82 to obstruct the downward travel of the hollow sleeve 92. As noted above, it is only when the device 10 is at least substantially horizontal in three dimensions that the ball 24 will sit in the bottom of the cavity 84 and allow the sleeve 92 to slide about the ball 24 to initiate the action (e.g., injection).

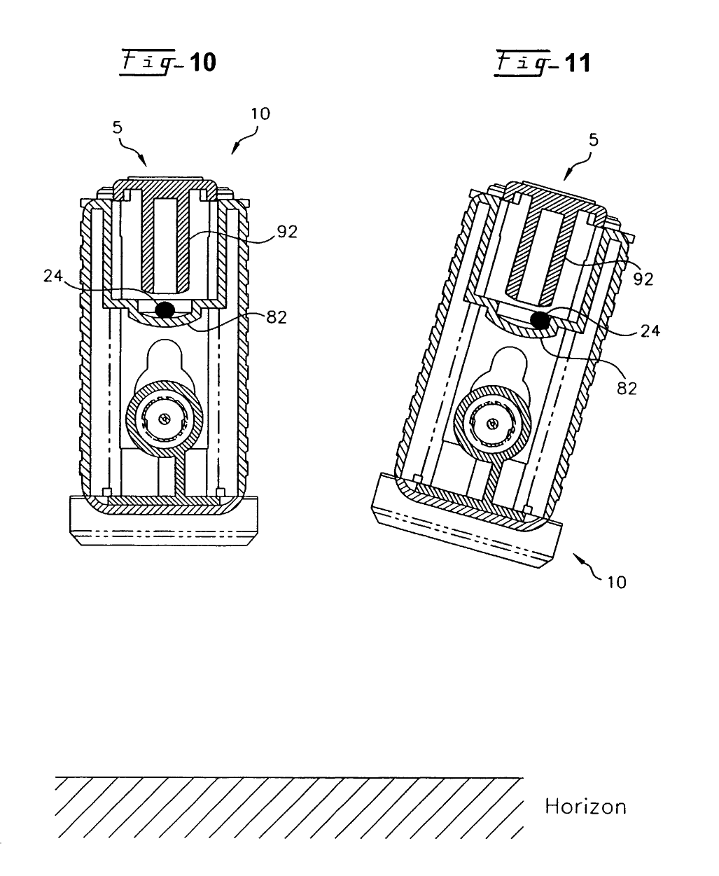

FIGS. 10 and 11 show a transverse sectional view of the delivery device 10 taken along line 10-10 of FIG. 4. As shown, the device 10 shown in FIG. 10 is horizontal and the device 10 shown in FIG. 11 is not horizontal. Since the device 10 in FIG. 11 is not in its preferred orientation (e.g., horizontal), the ball 24 is located at a side of the bowl 82 where it obstructs the downward travel of the sleeve. However, as shown in FIG. 10, when the device 10 is in its preferred orientation (e.g., horizontal) the ball 24 is directly under the sleeve 92 and in the sleeve's direction of travel (e.g., vertical). Here, the sleeve 92 can travel vertically over the ball 24 and initiate injection.

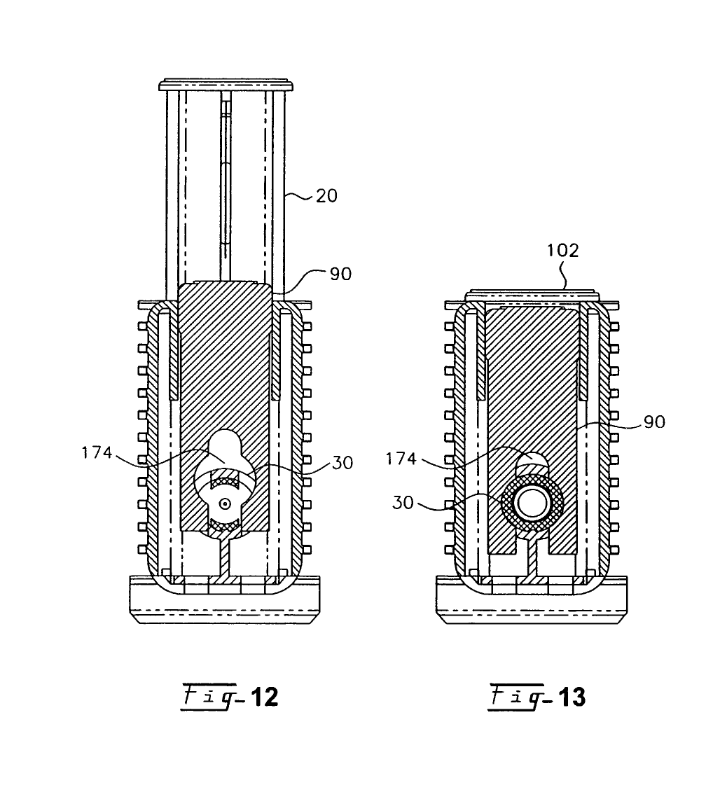

FIG. 12 is a transverse sectional view of the delivery device 10 taken along line 12-12 of FIG. 4. The actuator 90 of the trigger 22 is shown in its pre-initiating position prior to injection, that is, before the trigger 22 is pushed down and over the ball 24. When in its pre-injection position, the actuator 90 abuts the needle hub 30 and keeps the hub spring 32 in its compressed state. As shown in FIG. 12, the actuator 90 includes an aperture 174 sized to permit the hub 30 to slide through the aperture 174 when the aperture 174 is aligned with the hub 30. The aperture 174 and hub 30 are aligned when the trigger 22 is moved vertically to its initiating position, as shown in FIG. 13. It is important to repeat that in this example of the preferred embodiment, the trigger 22 cannot slide down to this initiating position unless the device 10 is substantially horizontal so that the ball 24 does not obstruct the downward travel of the sleeve 92.

The hub spring 32 is held in a compressed state (FIGS. 3 and 9) while the actuator 90 extending from the trigger 22 abuts the hub 30 holding the needle 33. The hub spring 32 is released when the trigger 22 is moved to its actuating position which slides the aperture 174 into alignment with the hub 30.

As can best be seen in FIGS. 1, 3, 9,14 and 15, the trigger 22 also includes a pin 176 having an extending finger 178 and a shoulder 180. The finger 178 extends through the opening 124 in the needle barrel 116 as the trigger 22 is guided into the trigger receiving cavity 58 such that the finger 178 is located in the needle barrel 116 when the aperture 174 of the actuator 90 is aligned with the hub 30. In this position, the finger 178 stops the forward movement of the hub 30 when the hub 30 is released from the actuator 90. Upon this release, the needle hub spring 32 longitudinally expands and biases the needle hub 30 toward the distal end of the shield 38 until the hub 30 reaches the finger 178. The hub 30, which is holding the injection needle 33, pushes the distal sharp end 142 of the needle 33 through the needle covering end 140 for instantly penetrating the skin of the person or intravenous administration set being injected. The penetration length of the needle 33 is preferably about 7 mm although any length that penetrates the skin (or intravenous administration set) and delivers the drug solution is sufficient.

As can best be seen in FIGS. 3, 9,14 and 15, the injection needle 33 is hollow and includes a central passageway 182 extending its distal sharp end 142 to the notch 144 located between the distal sharp end 142 and the needle's proximal end. When the injection assembly 114 is in its pre-injection position (FIGS. 3 and 9) the notch 144 is located in a central region of the needle barrel 116 between the needle hub 30 and the septum 36. However, when the injection needle 33 is pushed by the needle hub 30 to extend the distal sharp end 142 of the injection needle 33 beyond the shield 38, as shown in FIG. 14, the notch 144 of the injection needle 33 communicates with the injection chamber 119 of the septum 36, and therefore communicates with the drug solution via the drug inlet 164.

The central passageway 182 of the needle 33 provides a conduit for the drug solution to flow from the drug inlet 164 and injection chamber 119 into the patient or intravenous administration set being administered. In particular, when the notch 144 comes in to communication with the injection chamber 119, the pressure of the drug solution in the housing 52 is released. This allows the pressure to force the drug solution through the injection assembly 114 into the patient.

The delivery device 10 of this preferred embodiment includes another safety feature that protects a user or patient from unwanted contact with the needle 33. The shield 38 is arranged to permit injection if the shield 38 is pressed against the skin of the patient or an outer wall of the intravenous administration set. Otherwise, the shield 38 covers the distal sharp end 142 of the needle 33 to prevent external contact with the needle 33.

As shown in FIG. 14, when the trigger 22 is moved to its initiating position, the shoulder 180 of the trigger pin 176 pushes the hook 136 under the first retaining wall 146 and releases the grip of the hook 136. This movement frees the hook 136 from the first retaining wall 146 and permits the shield spring 40 to bias the shield 38 forward to a post-injection position where the needle covering end 140 extends further than the extended position of the distal sharp end 142 of the needle 33. However, the shield 38 is arranged so that its outer face 131 faces the patient and is pressed down onto the injection site prior to injection. If the outer face 131 is not pressed onto the injection site, then the shield 38 moves forward when the trigger 22 is moved to its initiating position and the shoulder 180 releases the hook 136 from the first retaining wall 146.

The septum 36 is attached to the shield 38 and moves forward with the shield 38. When the shield 38, and hence, the septum 36 slides forward, the injection chamber 119 of the septum 36 moves out of alignment with the drug inlet 164 and prevents fluid communication of the drug solution to the injection site. However, if the outer face 131 of the shield 38 is positioned so that it faces the patient and is pressed down onto the injection site, the pressure of the shield 38 against the injection site will prevent the shield 38 from moving forward under the bias of the shield spring 40 to cover the injection needle 33. Therefore the delivery device 10 is arranged to inject the drug solution when the injection assembly 114 is activated and the shield 38 is pressed against the injection site.

FIG. 15 illustrates the relative positions of the needle 33, septum 36, and shield 38 upon completion of the drug solution delivery. Upon the end of delivery, the user stops pressing the shield 38 against the injection site. This causes the shield spring 40 to bias the shield 38 forward to an extended position and cover the distal sharp end 142 of the injection needle 33. The hook 136 of the shield's upper arm 132 abuts a second retaining wall 186 of the body 26 to stop the forward motion of the shield 38 at its extended position. The lower arm 134 of the shield 38 abuts and snap fits about an inwardly extending tab 188 of the housing 52 to lock the shield 38 in its extended position by preventing the shield 38 from retracting into the housing 52. Any subsequent force applied to the shield 38 will not move the shield 38, thus preventing the re-exposure of the injection needle 33.

As should be apparent from the foregoing, the drug delivery device 10 of the preferred embodiment provides a safe and efficient approach to injecting a drug solution into a patient. Included in the device 10 is a safety mechanism, including the trigger 22 that will initiate the action, for example, drug delivery, only when the device 10 is substantially horizontal in three dimensions. Therefore, in this example of the preferred embodiment, the safety mechanism ensures that injection cannot be initiated unless the mechanism is oriented substantially horizontal in all three dimensions.

It should be apparent from the aforementioned description and attached drawings that the content of the present application may be readily applied to a variety of preferred embodiments, including those disclosed herein. For example, the safety mechanism can be adapted to ensure that an action cannot be initiated unless the mechanism is in a preferred orientation (e.g., horizontal, vertical). That is, the mechanism can be used in a variety of other applications, such as, a switch. Further, the delivery device could be an injection device or a reconstitution and injection device. In addition, other biasing devices or pumps such as elastomeric O-rings or compressed gas may be used in place of the helical compression springs disclosed herein to bias the needle hub 30 or shield 38, as readily understood by a skilled artisan.

Also, while the inner rim 86 is shown as being generally circular in the drawings, it is understood that the shape of the inner rim 86, and the shape of the bowl 82 is not critical to the preferred embodiments of the invention. What is important to the understanding of the preferred embodiment of the invention shown in the drawings is that the shape of the cavity 84 of the bowl 82 permits the ball 24 to move within the cavity 84 and block an initiating movement of the hollow sleeve 92 when the premature activation prevention mechanism 5 is not in a desired orientation (e.g., vertical, horizontal). Accordingly, any shape of the cavity 84 or inner rim 86 that permits the ball to move as described is considered bowl-shaped or concave within the scope of the invention. Therefore the shape of the inner rim 86 could be circular, polygonal or any combination thereof as understood by a skilled artisan.

It is further appreciated that the present invention may be used to deliver a number of drugs. The term “drug” used herein includes but is not limited to peptides or proteins (and memetics thereof), antigens, vaccines, including DNA vaccines, hormones, analgesics, anti-migraine agents, anti-coagulant agents, medications directed to the treatment of diseases and conditions of the central nervous system, narcotic antagonists, immunosuppressants, agents used in the treatment of AIDS, chelating agents, anti-anginal agents, chemotherapy agents, sedatives, anti-neoplastics, prostaglandins, antidiuretic agents and DNA or DNA/RNA molecules to support gene therapy.

Typical drugs include peptides, proteins or hormones (or any memetic or analogues of any thereof) such as insulin, calcitonin, calcitonin gene regulating protein, atrial natriuretic protein, colony stimulating factor, betaseron, erythropoietin (EPO), interferons such as a,b or g interferon, somatropin, somatotropin, somastostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2, and analogues or antagonists thereof, such as IL-1ra, thereof; analgesics such as fentanyl, sufentanil, butorphanol, buprenorphine, levorphanol, morphine, hydromorphone, hydrocodone, oxymorphone, methadone, lidocaine, bupivacaine, diclofenac, naproxen, paverin, and analogues thereof; anti-migraine agents such as sumatriptan, ergot alkaloids, and analogues thereof; anti-coagulant agents such as heparin, hirudin, and analogues thereof; anti-emetic agents such as scopolamine, ondansetron, domperidone, metoclopramide, and analogues thereof; cardiovascular agents, anti-hypertensive agents and vasodilators such as diltiazem, clonidine, nifedipine, verapamil, isosorbide-5-mononitrate, organic nitrates, agents used in treatment of heart disorders, and analogues thereof; sedatives such as benzodiazepines, phenothiozines, and analogues thereof; chelating agents such as deferoxamine, and analogues thereof; anti-diuretic agents such as desmopressin, vasopressin, and analogues thereof; anti-anginal agents such as nitroglycerine, and analogues thereof; anti-neoplastics such as fluorouracil, bleomycin, and analogues thereof; prostaglandins and analogues thereof; and chemotherapy agents such as vincristine, and analogues thereof, treatments for attention deficit disorder, methylphenidate, fluoxamine, Bisolperol, tactolimuls, sacrolimus and cyclosporin.

Without further elaboration, the foregoing will so fully illustrate the invention that others may, by applying current or future knowledge, readily adapt the same for use under various conditions of service.

What is claimed is:

1. A safety device for initiating an action, comprising:
a housing;
a trigger within said housing, said trigger movable from a first position to an activation position, wherein when said trigger is in said activated position, said trigger initiates the action; and
an orientation unit in communication with said housing and said trigger, said orientation unit enabling movement of said trigger from said first position to said activated position only when said housing is in a predetermined three dimensional orientation.

2. The safety device of claim 1, said orientation unit including an indicator arranged for providing an indication that said housing is in the predetermined three dimensional orientation.

3. The safety device of claim 2, said orientation unit further including a cavity defined by an inner rim, said indicator freely moveable within said cavity in response to the orientation of said housing, said indicator moving to a desired position of said cavity as the indication that said housing is in the predetermined three dimensional orientation, and said trigger arranged to move to said activation position only when said indicator is in the desired position.

4. The safety device of claim 3, wherein said indicator is a ball having a transverse dimension.

5. The safety device of claim 4, wherein said trigger is slidingly located within said housing, said trigger further including a hollow sleeve sitting adjacent said inner rim of said cavity, said hollow sleeve having an inner transverse dimension greater than the transverse dimension of said ball, said sleeve arranged to slide about said ball only when said ball is in the desired position enabling said trigger to initiate the action.

6. The safety device of claim 5, wherein said cavity is concave.

7. The safety device of claim 1, the action including ejecting a fluid from said housing into an injection site, and further comprising an injector assembly initiated by said trigger for providing the action.

8. The safety device of claim 7, said injector assembly including:
a reservoir located within said housing, said reservoir being arranged to hold the fluid;
a hollow injector within said housing, said injector arranged for providing fluid communication between said reservoir and the injection site; and
a piston slidingly located within said housing, said piston adapted to provide a pressure increase on the fluid in said reservoir.

9. The safety device of claim 8, the hollow injector comprising an injection needle, said needle having an opening, an interior and a tip, said opening arranged to provide fluid communication between said interior and said reservoir; and
a hub holding said needle, said hub being arranged for moving said needle from a first position where said opening in said needle is not in fluid communication with said reservoir to a second position where the opening in said needle is in fluid communication with said reservoir.

10. The safety device of claim 9, further comprising a pump located between said housing and said hub, said pump being arranged to create a bias, said actuator initiating the action by releasing the bias and allowing said pump to push said hub and move said needle from said first position to said second position, said trigger further including a pin slidingly located within said housing to prevent said hub from moving said needle beyond said second position.

11. A fluid delivery device, comprising:
a housing arranged for injecting a fluid into an injection site;
a trigger within said housing, said trigger movable from a first position to an activation position, wherein when said trigger is in said activated position, said trigger initiates the action of injecting the fluid;
an orientation unit in communication with said housing and said trigger, said orientation unit enabling movement of said trigger from said first position to said activated position only when said housing is in a predetermined three dimensional orientation; and
an injection assembly within said housing and in communication with said trigger, said injection assembly held under a releasable bias and arranged to expel the fluid from said delivery device upon release of the bias.

12. The fluid delivery device of claim 11, said orientation unit including an indicator arranged for providing an indication that said housing is in the predetermined three dimensional orientation.

13. The fluid delivery device of claim 12, said angular orientation unit further including a cavity defined by an inner rim, said indicator freely moveable within said cavity in response to the orientation of said housing, said indicator moving to a desired position of said cavity as the indication that said housing is in the predetermined three dimensional orientation, and said trigger arranged to move to said activation position only when said indicator is in the desired position.

14. The fluid delivery device of claim 13, said trigger including an actuator and a hollow sleeve, said hollow sleeve sitting adjacent said inner rim of said cavity, said hollow sleeve having an inner transverse dimension greater than a transverse dimension of said indicator, said sleeve arranged to slide about said indicator only when said indicator is in the desired position of said cavity enabling movement of said actuator to a position for releasing the bias and permitting said injection assembly to eject the fluid.

15. The fluid delivery device of claim 14, wherein said indicator is in the desired position of said cavity when said hollow sleeve is substantially vertical.

16. The fluid delivery device of claim 14, wherein said indicator is a ball.

17. The fluid delivery device of claim 11, further comprising a safety tab engageable with said trigger, said tab arranged to prevent movement of said trigger when said tab is engaged with said trigger.

18. The fluid delivery device of claim 11, further comprising a piston slidingly located within said housing, said piston arranged to place the fluid under a pressure adapted to be released upon movement of said trigger to the activation position.

19. The fluid delivery device of claim 18, said housing including a channel, said channel arranged to provide fluid communication between said piston and said injection assembly.

20. The fluid delivery device of claim 11, said injection assembly including a pump, a tube and a reservoir, said reservoir holding said fluid, said pump being arranged to push said tube to provide fluid communication with said reservoir and expel said fluid upon release of the bias.

21. The fluid delivery device of claim 20, wherein said pump is a compression spring.

22. The fluid delivery device of claim 11, said injection assembly including:
a reservoir located within said housing, said reservoir holding the fluid;
a hollow needle slidingly located within said housing, said needle having an opening, an interior and a tip, said opening arranged to provide fluid communication between said interior and said reservoir; and
a hub holding said needle, said hub being arranged for moving said needle from a dry position where said opening in said needle is not in fluid communication with said reservoir to a wet position where the opening in said needle is in fluid communication with said reservoir.

23. The fluid delivery device of claim 22, further comprising a pump located between said housing and said hub, said pump being arranged to create said bias, and upon release of said bias, to push said hub to move said needle from said dry position to said wet position.

24. The fluid delivery device of claim 23, wherein said pump is a compression spring.

25. The fluid delivery device of claim 22, further comprising a shield arranged to conceal said tip of said needle when said needle is in said dry position.

26. The fluid delivery device of claim 25, further comprising a pump located between said housing and said shield, said pump being arranged to bias said shield beyond said tip of said needle and conceal said tip upon completion of the ejection of the fluid.

27. The fluid delivery device of claim 26, said trigger further including a pin slidingly located within said housing to activate said pump for biasing said shield beyond said tip.

28. The fluid delivery device of claim 26, wherein said pump is a compression spring.

29. The fluid delivery device of claim 22, said trigger further including a pin slidingly located within said housing to prevent said hub from moving said needle beyond said wet position.

30. The fluid delivery device of claim 22, further comprising a septum providing fluid communication between said reservoir and said needle.

31. A method of ejecting a fluid from a delivery device, the delivery device including a housing, a trigger, an orientation unit, and an injection assembly, the housing arranged for injecting a fluid into an injection site, the trigger located within the housing, the orientation unit being in communication with the housing and the trigger, the injection assembly located within the housing and in communication with the trigger, the injection assembly held under a releasable bias and arranged to eject the fluid from the delivery device upon release of the bias, the method comprising:
(a) enabling movement of the trigger from a first position to an activation position only when the housing is in a predetermined three dimensional orientation;
(b) activating the injection assembly by releasing the bias by the movement of the trigger; and
(c) providing an egress for ejecting the fluid from the delivery device.
